(19) United States
(12) Patent Application Publication
(10) Pub. No.: US 2007/0065841 A1
(43) Pub. Date: Mar. 22, 2007

(54) Methods and compositions for the removal of nucleic acid amplification inhibitors

(76) Inventors: John M. Dunbar, Santa Fe, NM (US); Peter C. Stark, Los Alamos, NM (US); Thomas W. Robison, Los Alamos, NM (US); Raquel A. Lazarin, Milwaukee, WI (US)

Correspondence Address: LOS ALAMOS NATIONAL SECURITY, LLC, LOS ALAMOS NATIONAL LABORATORY, PPO. BOX 1663, LC/IP, MS A187, LOS ALAMOS, NM, 87545, US

(21) Appl. No.: 11/404,235

(22) Filed: Apr. 14, 2006

Related U.S. Application Data
(60) Provisional application No. 60/671,771, filed on Apr. 16, 2005.

(51) Int. Cl. C12Q 1/68; C07H 21/04

(52) U.S. Cl. 435/6; 536/25.4

(57) ABSTRACT

An improved method for preparing nucleic acid extracts from environmental samples contaminated by polymerase inhibitors such as humic and fulvic acids is provided. The methods of the invention utilize chemical compositions capable of precipitating humic and/or fulvic acids from organic samples. The methods may be used in connection with the preparation of DNA and RNA extracts, thereby providing purified preparations suitable for amplification using PCR, RT-PCR, and other nucleic acid amplification technologies. Reagent kits for preparing a purified nucleic acid-containing extract from an environmental sample of soil, fluid, or organic particles, using the chemical compositions of the invention are also provided.

### RELATED APPLICATIONS

This patent application claims the benefit of the filing date of U.S. Provisional patent application No. 60/671,771 filed Apr. 16, 2005 under 35 U.S.C. 119(e).

### STATEMENT AS TO RIGHTS TO INVENTIONS MADE UNDER FEDERALLY SPONSORED RESEARCH OR DEVELOPMENT

This invention was made with government support under Contract No. W-7405-ENG-36 awarded by the United States Department of Energy to The Regents of The University of California. The government has certain rights in this invention.

### BACKGROUND OF THE INVENTION

Forensic application of molecular techniques in environmental microbiology requires efficient extraction and purification of nucleic acids. Numerous DNA extraction methods have been developed and evaluated for acquiring genetic material from microorganisms present in soils and sediments, aerosols, water and other aqueous samples, as indigenous species or as organisms intentionally introduced to the environment. Humic and fulvic acids are found in water, air-borne organic materials, soils and sediments, and inhibit enzymatic (polymerase) activities characteristic of nucleic acid amplification techniques such as the polymerase chain reaction (PCR).¹⁻⁶

Humic and fulvic acids (HAs) are naturally occurring, polyelectrolytic, heterogeneous, organic substances that are generally dark brown in color, of relatively high molecular weight and, typically, resistant to degradation.⁹ They contain multiple functional groups such as phenolic and carboxylic moieties as well as hydrophobic components such as aliphatic or aromatic moieties.

Soils and sediments containing high organic carbon content also contain high levels of humic and fulvic acids. Humic acid concentrations from soil extractions vary according to soil/substrate types and for extraction methods, and in general, are found at concentrations ranging from 100-5000 mg/L. Accordingly, nucleic acid preparations extracted from soil and sediment can contain high levels of humic and fulvic acids, which in turn inhibit the amplification of the extracted nucleic acids.⁷ For example, standard PCR reactions have been inhibited by as little as 10 ng of humic acid. Additionally, the lysis and extraction method affects the quantity and quality of DNA recovered.⁵⁻⁷ The type of extraction method used may also preferentially yield DNA from one species relative to another species, and may also influence the amount of inhibitory substances co-extracted.⁸

Other problematic matrices for DNA extraction include: clinical samples (stools, blood, sputum, etc.), domesticated animal manures, sewage, plant materials with high polysaccharide concentrations and samples with high transition and alkali earth metal concentrations.

Many methods describing PCR inhibitor removal from samples containing nucleic acids subsequently used for PCR amplification have been described and include: electroelution,^{10,11} polyvinylpolypyrrolidone spin columns,^{4,12,13} serial dilution of extracts,⁵ addition of bovine serum albumin to extracts,¹⁴ pre-extraction and removal of the humics followed by cell lysis/DNA extraction,¹⁵ gel filtration resins,^{16,17} chemical coprecipitation/flocculation with transition metal oxy/hydroxides,¹⁸ hexadecyltrimethylammonium bromide preparations,¹⁹ hydroxyapatite column purification,²⁰ cesium chloride density centrifugation,³ ion exchange and size exclusion chromatography,^{3,8} and even agarose gel electrophoresis coupled with excision and further DNA extraction from the gel matrix.¹⁹

Additionally, kits designed and marketed specifically for DNA extraction from sample matrices containing PCR inhibitors are commercially available. Currently, the best commercially available solution for isolating clean DNA from humic acid contaminated samples is the “UltraClean Soil DNA Kit” marketed by MoBio Laboratories, Inc. This kit utilizes aluminum sulfate to co-precipitate HAs. However, there are some soils which exceed the kit's capabilities, the procedure is complex, and performance is limited (for example, sample dilution may be required, thereby reducing the number of DNA copies available for amplification). Other commercial kits include the SoilMaster™ DNA Extraction kit marketed by Epicentre^{21,22}, GeneReleaser™ and Maximator® kits by Bioventures Inc.²³, and QIAamp® by Qiagen^{24,25}. However, many of these methods are time consuming, require a high level of technical expertise, and generate poor nucleic acid yield and quality.

Phenacylthiazolium bromide (PTB) has been identified as an advanced glycation end product (AGE) crosslink breaker and AGE inhibitor.^{26,27} There are some structural and chemical similarities between AGE's and HAs, notably the presence of carbonyl, dicarbonyl and hydroxyl moieties. PTB has also been shown to allow PCR amplification of extinct sloth DNA from ancient coprolites and other ancient skeletal remains by removal of PCR inhibitors.^{28,29} Considering these results, PTB was tested as a candidate for removal of humic acids from soil samples containing DNA of interest. PTB is an effective remover of humic acids and allows PCR in samples that would otherwise require more complicated and time consuming inhibitor removal techniques described previously; however, PTB's solubility in aqueous solutions is limited (<1.0M). Further, photo, thermal and solution phase storage stability are less than optimal while commercial availability of PTB is limited to a single supplier at a significant cost.

These issues reveal a need for a simple, reliable and inexpensive method in which nucleic acid amplification inhibitors may be removed from soil and other environmental extracts containing DNA or RNA of interest. The ideal method should also accommodate nucleic acid extraction from species at trace concentrations, without considerable material loss, in order to facilitate forensic investigations.

### SUMMARY OF THE INVENTION

The invention provides an improved method for preparing nucleic acid extracts from environmental samples contaminated by polymerase inhibitors such as humic and fulvic acids. The presence of such polymerase inhibitors in nucleic acid extracts impedes the amplification of nucleic acids by PCR, RT-PCR, and various isothermal DNA amplification methods, all of which utilize polymerase enzymes in the amplification process. The methods of the invention utilize chemical compositions capable of precipitating humic and/or fulvic acids from organic samples. The methods may be used in connection with the preparation of DNA and RNA extracts, thereby providing purified preparations suitable for amplification using PCR, RT-PCR, and other nucleic acid amplification technologies.

In one embodiment, a method for preparing a purified nucleic acid-containing extract from an environmental sample of soil, fluid, or organic particles is provided. Briefly, the method comprises preparing an aqueous nucleic acid-containing extract from the environmental sample; adding to the extract at least one PIR compound, preferably selected from the group consisting of thiamine hydrochloride, thiamine pyrophosphate and pyridoxamine; mixing the PIR compound(s) into the extract for a time sufficient to precipitate humic acids, fulvic acids and other insoluble contaminants contained in the extract, thereby generating insoluble precipitate and soluble fractions, and isolating the soluble fraction therefrom.

In another embodiment, the above method further comprises subjecting the isolated soluble fraction to further nucleic acid purification, including for example, purification via one or more nucleic acid precipitation and wash steps, ultrafiltration, and the like.

Such purification steps may improve the amplifiability of the nucleic acids in the extract using PCR and other amplification methods.

The methods of the invention are particularly useful in preparing extracts from humic-containing samples, such as soil samples, water or other liquid sample (e.g., blood), aerosol samples (e.g., particulate material captured from the air on a filter or other capture material), as well as various other organic or environmental sample types, including stool samples, sewage samples, plant materials, and the like.

### BRIEF DESCRIPTION OF THE DRAWINGS

FIG. 1. PIR efficiency based on HA reduction factor in decreasing order as PDA>>THI/BRT>DTPB>PTB>AAMG.

FIG. 2. PIR efficiency based on 0 and decade of dilution in decreasing order as PDA>BRT>THI>PTB>AMG.

FIG. 3. PIR ability of 1M PTB solutions is slightly affected by lighting conditions and low temperature storage.

FIG. 4. Thiamine's PIR reduction factor is affected by length of storage, but not considerably by temperature or lighting conditions.

FIG. 5. Thiamine's effectiveness as PIR is relatively not impacted by storage conditions but by length of storage.

FIG. 6. 1 day and 1 month old 1M solutions of BRT have similar PIR ability Regardless of storage conditions.

FIG. 7. 1M phenacylthiazolium bromide removes humic materials but often requires further dilution to complete PCR inhibition removal sufficiently to achieve PCR.

FIG. 8. Humic acid reduction factors for PIR's on soil extracts reveal PDA and THI generate samples amenable to PCR with minimal dilution required.

FIG. 9. Efficiency values for HA removal by PIR's (THI and PDA) show similar Performance in soil extracts/samples.

FIG. 10. Effect of PIR compound on soil DNA extraction. Soil was homogenized 2 minutes, the PIR compound was added to appropriate tubes, and all tubes were centrifuged at the indicated speeds. As shown, even low speed spins (1000 rpm) with the PIR compound present can effectively remove particulates, indicating that this step can be replaced by coarse filtration.

FIG. 11. Crude extracts of 5 soils. Shown are untreated and treated (i.e. PIR compound added) samples of each soil.

FIG. 12. Effect of PIR10 (Thiamine) on purity of soil DNA extracts as indicated by specific amplification of B. anthracis pag gene fragment. All soil samples were spiked with 100 ng of B. anthracis Sterne DNA (˜10⁷ cell equivalents) prior to extraction, yielding a predicted final concentration of ≦10⁴ cell equivalents/μl soil extract. The expected size of the pag amplicon is 134 bp. The numbers above lanes indicate the 10-fold dilution factor, where “0” is undiluted DNA extract.

FIG. 13. Effect of PIR10 (Thiamine) on purity of soil DNA extracts as indicated by amplification of bacterial 16S rDNA. The expected size of the 16S rDNA amplicon is ˜931 bp. The numbers above lanes indicate the 10-fold dilution factor, where “0” is undiluted DNA extract.

FIG. 14. Effect of ultra-filtration on purity of soil DNA extracts. Soil DNA extracts from untreated or PIR-10 (Thiamine) treated soil samples were ultra-filtered using Microcon 100 filters (100 kDa molecular weight cutoff), then serially diluted and tested by PCR amplification of 16S rDNA. The Microcon 100 filters retain large molecules (e.g. DNA) but allow small molecules (e.g. metal ions, salts, and low molecular weight fulvic acids) to pass through.

FIG. 15. Effect of PIR10 (Thiamine) on purity of DNA extracts from soils C and E as indicated by amplification of bacterial 16S rDNA. The expected size of the 16S rDNA amplicon is ˜931 bp. The numbers above lanes indicate the 10-fold dilution factor, where “0” is undiluted DNA extract.

FIG. 16. Chemical structure of thiamine hydrochloride.

FIG. 17. Chemical structure of thiamine pyrophosphate.

FIG. 18. Chemical structure of pyridoxamine.

### DETAILED DESCRIPTION

The invention relates to methods and compositions for the efficient removal of compounds, including without limitation humic and fulvic acids, which inhibit the activity of polymerases used in nucleic acid amplification techniques, such as PCR, from soil, liquid and aerosol samples containing nucleic acids and/or cells containing nucleic acids. Accordingly, the invention provides a method for preparing a nucleic acid-containing extract in which humic and fulvic acids and other polymerase inhibitors are removed to an extent that permits efficient nucleic acid amplification from the extract. The method is useful in preparing extracts from humic-containing samples, such as soil samples, water or other liquid sample (e.g., blood), aerosol samples (e.g., particulate material captured from the air on a filter or other capture material), as well as various other organic or environmental samples types, including stool samples, sewage samples, plant materials, and the like.

The methods of the invention utilize chemical compositions capable of precipitating humic and/or fulvic acids, and/or other polymerase inhibiting compounds, from organic samples. Such compositions are herein referred to as “Polymerase Inhibitor Removal”, “PCR Inhibitor Removal” or “PIR” compounds. The methods may be used in connection with the preparation of DNA and RNA extracts, thereby providing purified preparations for the amplification of both DNA and RNA, using any nucleic acid amplification technique which utilizes a polymerase. Such amplification methods include, for example, the widely used polymerase chain reaction (PCR), RT-PCR, as well as various isothermal amplification methods known in the art.

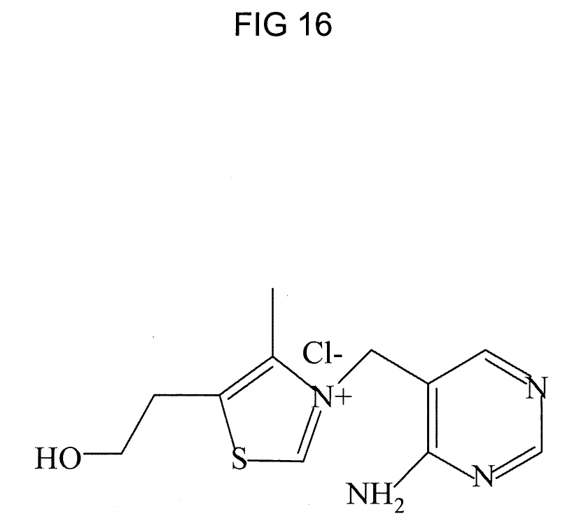

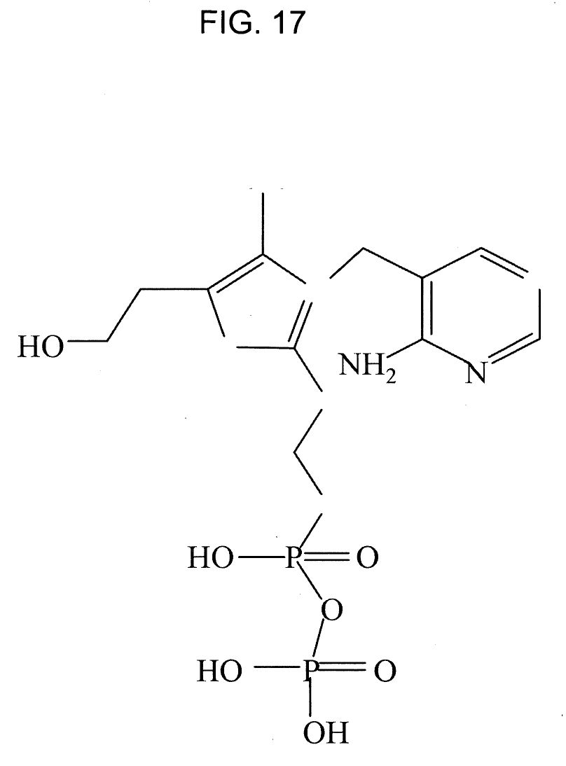

In the practice of the method of the invention, a PIR compound is added to a sample from which DNA or RNA is to be extracted for subsequent amplification. The PIR compound may be added to crude extracts of nucleic acid-containing samples, samples which have undergone an initial filtration step, or to the samples directly prior to any cell lysis and nucleic acid extraction. Inhibitory organic contaminants such as humic and fulvic acids are precipitated by the PIR compound and are removed from the extract, resulting in an extract containing PCR-amplifiable nucleic acids. In one embodiment, thiamine (vitamin B1) is used as the PIR compound. Both thiamine hydrochloride and thiamine pyrophosphate are particularly effective PIR compounds. The structures of thiamine hydrochloride and thiamine pyrophosphate are shown in FIGS. 16 and 17, respectively.

As Described in Example 2, infra, thiamine performed exceptionally well in DNA extraction and PCR amplification tests. More specifically, thiamine improved the purity of all soil DNAs tested, as indicated by improved detection limits and/or higher yields in PCR product. Purity of DNA samples was improved by two orders of magnitude using this PIR compound in DNA extraction protocols. An ultrafiltration step further improved the purity of all DNA samples tested by another order of magnitude.

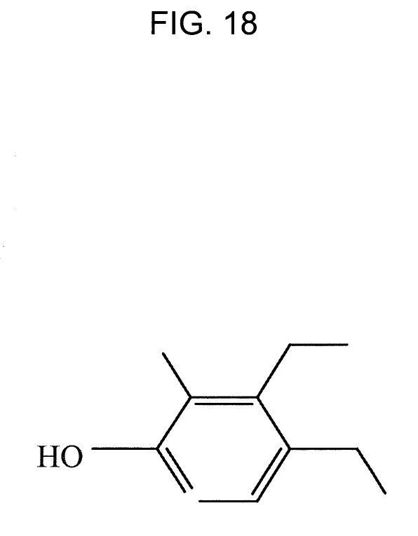

In another embodiment, pyridoxamine (PDA) is used as the PIR compound. PDA is a known compound, the structure of which is shown in FIG. 18. PDA demonstrates superior PIR activity, rapid kinetics, and high stability (see Example 1, infra).

The above PIR compounds of the invention are preferred and have proven particularly capable of effectively removing humic acids from soils and providing extracts which yield sufficient quantities of amplified DNA using PCR. In addition, these preferred PIR compounds have demonstrated desirable solubility, stability and/or reaction kinetics. See Examples, infra. Other compounds showing PIR activity are also described herein, see infra. Some of these, e.g., phenylthiazolium bromide (PTB), demonstrate the capacity to effectively remove humic acids, but may otherwise exhibit poor solubility, stability and/or reaction kinetics in relation to the preferred PIR compounds of the invention.

The PIR compounds of the invention may be used as described in the Examples, infra. As will be appreciated by those skilled in the art, variations of the protocols shown herein may be incorporated into the methods of the invention, including for example, the use of various DNA amplification strategies, the use of various buffered solutions, enzymes, and extraction protocols. For illustration, the DNA extraction methods of the invention generally comprise the preparation of a nucleic acid containing extract from an environmental samples such as soil, water, air, etc., in which the extract is mixed with at least one PIR compound for a time sufficient to precipitate humic acids, fulvic acids and other contaminants. The resulting insoluble precipitate is isolated by centrifugation and/or filtration, using standard techniques, and the nucleic acid containing supernatant or fraction is removed or otherwise isolated. This extract may be further subjected to ultrafiltration and washing, as is well known in the art.

In one embodiment, cells within the environmental sample are lysed in a buffered aqueous solution, as is well known (i.e., sodium phosphate buffer), and cellular debris and other insoluble materials are separated. The PIR compound may be added to this crude extract or to semi-purified extracts, in solution at ambient temperature, in order to precipitate amplification-inhibiting contaminants. The PIR compound may be included in the extraction buffer or, preferably, added following cell and/or microbial particle lysis, or following separation of cellular debris and insoluble materials and/or initial purification. After the formation of a precipitate (insoluble fraction), the insoluble fraction containing the precipitated contaminants is separated from the soluble, nucleic acid-containing fraction, and the soluble fraction subjected to standard nucleic acid purification, ultrafiltration and/or washing steps. The purified nucleic acid containing solution may then be used for nucleic acid amplification.

As illustrated in detail in Example 2, infra, a preferred method for extracting nucleic acids free of humic and/or fulvic acid contaminants involves the use of “beadbeater” homogenization technology to lyse cells and extract the nucleic acids. More specifically, for example, a soil sample is added to a tube containing microbeads of variable size, typically two to three different sizes (e.g., 0.1 mm and 0.5 mm) in a sterile extraction buffered solution (e.g., TE, pH 8.0+0.1% SDS). The mixture is then homogenized to lyse cells within the sample, using a beadbeater device, as is well known. A PIR solution, comprising the PIR compound dissolved in a buffered solution, such as a sodium phosphate buffered solution, is then added and mixed with the homogenized material, and precipitated contaminants are removed by centrifugation (e.g., 12,000 rpm; 30 seconds). The nucleic acid-containing supernatant fraction is removed. This fraction may be used directly for PCR amplification of nucleic acids within the sample or, preferably, subjected to one or more filtration and purification steps. As the results presented in Example 2 show, further purification of the extract by ultrafiltration yields higher quality nucleic acids for PCR amplification. Typically, then, nucleic acids are precipitated from the nucleic acid-containing supernatant fraction, pelleted, and the pellet resuspended in Tris-EDTA, and subjected to ultrafiltration (e.g., Micron 100 filter). This step may be combined with washing, reprecipitation and resuspension steps in order to improve purification of the nucleic acids, as is well known. Dilution of purified nucleic acid-containing solutions may be required prior to PCR or other nucleic acid amplification methods.

The preferred protocols of the invention add PIR compounds in solution. An alternative technique involves attaching the PIR compound to a solid phase, such as glass or ceramic beads, which may be used directly in the homogenization step. PIR compounds may be used alone or in combination. Combining two or more PIR compounds in the extraction procedure may yield better results when used to extract nucleic acids from mixed samples and samples from diverse locations. For example, across the planet, humic acids vary from place to place and from soil type to soil type. Utilizing a mixture of PIR compounds may therefore enable broader applicability of kits utilizing the methods of the invention. Simple screening of PIR compounds against humics from different geographic locations and different sample types, for example, should enable the development of ideal PIR compounds or mixtures of PIR compounds for specific applications.

The methods of the invention may be applied to the preparation of RNA and DNA extracts from samples containing eukaryotic and prokaryotic cells, microbes, fungi, viruses, and the like. Numerous DNA, RNA and combined nucleic acid extraction techniques are known, and commercially available kits for this purpose are widely available. Various methods for lysing cells and viral particles are well known and routinely used in the art. For example, various physical methods of cell/particle breakage include mechanical cell disintegration (crushing and grinding, wet milling, ultrasonics, hydraulic shear, freeze pressure), liquid or hydrodynamic shear (French press, Chaikoff press, homogenizers, wet mills, vibration mills, filters, ultrasonic disintegration) and solid shear (grinding, Hugues press). Chemical methods for disintegration include those which target the cell wall and/or cytoplasmic membrane, viral capsid and envelope structures, and the like. So-called “beadbeating” technology uses vigorous agitation of cells and particles in a sample in the presence of glass, ceramic, or metallic (e.g., titanium) microbeads (typically between 0.1 mm and 1.0 mm) to mechanically disrupt cells and viral particles in order to release nucleic acids contained therein. Various buffers for use in such extraction methods are known, and include, for example, Tris-EDTA buffers for DNA extraction and guanididium thiocyanate buffers for RNA extraction.

Nucleic acid extraction and purification kits and buffers are widely available from many suppliers, including without limitation, Amersham, Invitrogen, Ambion, Applied Biosystems, Eppendorf, Genetix, Promega, Novagen, Qiagen and Strategene. In principle, any of these extraction methodologies may be combined with the use of PIR compounds in situations where nucleic acids must be amplified from humic and/or fulvic acid containing samples. As will be readily appreciated by those skilled in the art, introduction of one or more PIR compounds to the appropriate extraction step can be used to precipitate humic and/or fulvic acids from such samples, thereby yielding purified DNA or RNA preparations capable of being efficiently amplified by PCR, RT-PCR and other amplification technologies.

Purified, contaminant-free DNA or RNA is a prerequisite for successful microbial assessment of environmental samples and greatly reduces the occurrence of false negatives in screening assays which rely on the detection of signature nucleic acid sequences in amplified nucleic acid preparations. The methods of the invention are simple, fast, and do not require highly trained technical personnel. The invention's approach to purifying extracts for nucleic acid amplification has been validated as superior to conventional purification methods by several orders of magnitude. Thiamine, pyridoxamine, and phenylthiazolium bromide out-perform the best commercial soil DNA extraction kit by 10 to 100 fold, and improve PCR amplification 10 to 10,000 fold over untreated samples. For example, pyridoxamine can remove concentrations of humic acids which exceed natural levels in the environment.

In addition to their PIR activity, the PIR compounds described herein may also be useful in other applications in which it is desirable to break and/or inhibit AGE crosslinks, potentially including human age-related disease conditions characterized by the presence of AGE-protein crosslinks, including for example, cardiovascular diseases (e.g., decreased myocardial elasticity) and various complications arising from diabetes (e.g., nephropathy, retinopathy and neuropathy).³²

Other applications for the methods and PIR compounds of the invention include the purification of humic and/or fulvic acids from waste water treatment systems subjected to these contaminants, thereby improving the treatment system's efficacy for other contaminants.

The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001.

The invention further provides reagent kits for extracting, isolating and/or purifying nucleic acids, which utilize the PIR compounds and methods of the invention. The kits of the invention may contain various reagents and materials used in the practice of the methods of the invention, including without limitation, reagents and materials for extracting and purifying nucleic acids from environmental samples, sample collection devices, tubes, homogenization materials, extraction tubes or cartridges, solutions and buffers, and DNA/RNA amplification reagents. Kits may also include control samples, materials useful in calibrating amplification, and containers, tubes, and the like in which reactions may be conducted. Kits may be packaged in containers, which may comprise compartments for receiving the contents of the kits, instructions for conducting the extractions and amplifications, etc. In one embodiment, a reagent kit for preparing a purified nucleic acid-containing extract from an environmental sample of soil, fluid, or organic particles, comprises: (a) at least one PIR compound slected from the group consisting of thamine hydrochloride, thiamine pyrophosphate, 1,3-dithiazole-2-propanone dibromide, and pyridoxamine; and, (b) instructions for using the PIR compound in accordance with the method of the invention.

The invention is now further described by way of the following Examples which illustrate the synthesis and initial characterization of various candidate PIR compounds, and the characterization of PIR compound performance across a spectrum of parameters, including humic acid removal, PCR amplified DNA quantity and purity, and PIR compound solubility, stability and kinetic properties.

### EXAMPLES

### Example 1

### Synthesis and evaluation of PTB Derivatives, Analogues and Other Age-Inhibitory Compounds for Capacity to Remove Nucleic Acid Amplification Inhibitors

Materials and Methods

Materials:

Aminoguanidine; 2-bromo-4′-nitroacetophenone; 1,3-dichloroacetone; 1,3-dibromo-2-propanone; thiazole; anhydrous toluene; and (3-chloropropyl)trimethoxysilane were obtained from Acros Organics. Palladium, 10% wt on carbon; 2-bromoacetophenone; 1,3-dichloroacetone; chloroacetone; chloromethyltriethoxy silane; propionyl bromide; triethylamine; and silica gel 60-200 mesh were obtained from Aldrich Chemicals. Pyridoxamine dihydrochloride was obtained from MP Biomedicals. Thiamine pyrophosphate chloride was obtained from ICN Biomedicals. Glass beads were obtained from BioSpec Products. Humic acid (lot #H06N27) was obtained from Alfa Aesar. Humic and Fulvic acid standards (1S101H, 1S101F) were obtained from the International Humic Substances Society. Thiamine hydrochloride, anhydrous methanol, ethanol, propanol, OPTIMA grade concentrated sulfuric acid, tris-acetate EDTA and 35% hydrogen peroxide were obtained from Fisher Chemicals.

Methods:

Fluorimetric Determination of Humic Acids in Solution:

Aqueous samples of humic solutions were interrogated via fluorimetry on a Jobin Jvon Spex Fluorolog II with an excitation wavelength of 471 nm, an emission wavelength of 529 nm, with slits @ 10 nm bandpass, pmt bias of 950V, 1 second integration per reading and 10 readings per sample or <0.1% RSD per sample. Humic solutions were in 1×TAE buffer at RT with pH˜7. Standard solutions generating a linear fluorimetric response were as follows: IHSS HA 1S101H 0, 1, 2, 4, 6, 8, 10 mg/L; Sigma Aldrich humic acid (HA) 0, 1, 2, 4, 6 mg/L and; Alfa Aesar humic acid 0, 1, 2, 3, 4 mg/L. Typically, the Sigma HA generated a Fluorimetric response a factor of 2.2 greater intensity and the Alfa Aesar generated a Fluorimetric response a factor of 7.5 greater intensity within this linear region for equivalent concentrations of material. Samples from PIR experiments utilizing these materials were diluted, using 1×TAE, to concentrations falling within the linear response region representative of the humic material used.

Infrared Spectroscopy of PIR Compounds, HA/PIR Precipitates and Humic Acid Materials:

ATR-FTIR spectra of PIR compounds were acquired using a Nicolet Avatar 360 FT-IR and an Omni SamplilR Ge ATR attachment. FT-IR spectra of humic acid/PIR compound precipitates were acquired using a Nicolet Avatar 360 FT-IR with transmission baseplate. KBr pellets for humic acid/PIR compound precipitates and humic acid materials were prepared in a SPEX 3630 X-Press.

Humic acids and precipitates from reactions between humic materials and PIR compounds were acquired via centrifugation as part of the PIR protocols. Representative samples of glass substrates with, and without, silane and/or tethered PIR compounds were acquired during synthetic procedures. Representative samples were mixed with potassium bromide and pressed into pellets for subsequent interrogation via IR spectroscopy. Typically, 5-10 mg of PIR/HA precipitate was compounded with 50-100 mg of KBr using a mortar and pestle. The compounded mixture was transferred to a pellet mold and pressed for 90 seconds at 9 tons pressure. The pellet was then used to acquire the IR spectrum of the PIR/HA precipitate.

IR spectra of humic materials and review of literature regarding humics generated the wavenumber assignments of: 2920 aliphatic C—H stretch; 1725 carboxyl and ketone carbonyl stretch; 1620 aromatic C═C and conjugated carbonyl C═O; 1400 symmetrical COO⁻, OH deformation and C—O stretch of phenol groups; 1200 C—O stretch, OH deformation of COOH; 1050 carbohydrate or alcoholic C—O.

NMR Spectroscopy of PIR Compounds:

C13 and H NMR spectra for synthetically derived PIR compounds, and some PIR compound precursors, were acquired using a Bruker multi-element NMR.

Synthesis and Characterization of PIR Compounds:

Phenacyl Thiazolium Bromide (PTB):

Thiazole (2.5 g) and phenacyl bromide (5.85 g) were combined in 30 mL of ethanol to generate 1M solutions, respectively. Under reflux conditions for 2.5 hours generated 5.8 g crude PTB (˜75% yield). Recrystallization in 90% hot ethanol:10% nanopure water yielded 4.3 g pure PTB (51% overall yield) with a melting point of 223° C.

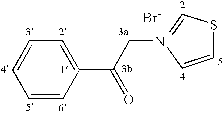

C-13 NMR peak assignments:

C2-161.5, C4-138.3, C5-126.1, C3a-60.4, C3b-190.5, C1′-134.5, C2′-128.1, C3′-129.0, C4′-133.5, C5′-129.0, C6′-128.1

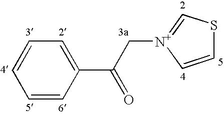

H NMR peak/shift assignments:

2-10.22 ppm, 4/5-8.45/8.55 ppm, 3a(2H)-6.50 ppm, 2′/6′&3′/5′-7.65&8.05 ppm, 4′-7.80 ppm

Bromothiazole (BAT):

Thiazole (1.0 g) was dissolved in 75 mL cold ether to which propionyl bromide (1.61 g) was added dropwise with stirring immediately generating a yellow, fluffy precipitate (4.72 g, ˜74% yield) which was isolated by filtering while under dry N₂, washing with 100 mL cold, anhydrous ether and having a melting point of 112° C.

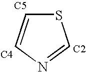

C-13 NMR peak assignments:

C2-156.4, C4-140.6, C5-123.5

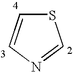

H NMR peak/shift assignments:

2-9.55, 3/4-8.00/8.15

4-amine-phenacyl thiazolium bromide (PTB-A):

2-bromo-4′-nitroacetophenone (1.5015 g, 0.08M) and 10% palladium on carbon (0.3141 g) were added to 75 mL of 95% ethanol in the presence of a catalytic amount of paratolune sulfonic acid (PTSA). The 4′-nitro group was reduced to an amine by the presence of 40 psi hydrogen gas for 24 hours with vigorous shaking in a PARR reaction vessel. The catalyst was removed from the reaction solution via filtration and the aminated phenone recovered after removal of the solvent via rotoevaporation. Thiazole (1 g, 0.39M) and 2-bromo-4′-aminoacetophenone (1.0387 g, 0.14M) were dissolved in 30 mL anhydrous ethanol and refluxed for 4 hours. Unpurified, 4-amine-phenacyl thiazolium bromide was recrystallized in 95% ethanol generating 0.5831 g purified PTB-A (14% overall yield).

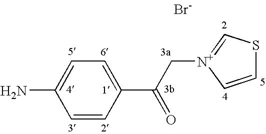

C-13 NMR peak/shift assignments:

C2-156.7, C4-138.3, C5-123.2, C3a-59.8, C3b-187.5, C1′-125.9, C2′-130.0,

C3′-110.6, C4′-130.0, C5′-110.6, C6′-161.3

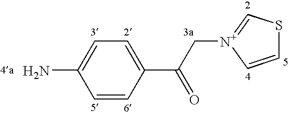

H NMR peak/shift assignments:

2(1H, s)-10.20 ppm, 4/5(1H, d)-8.40/8.50 ppm, 3a-6.30 ppm(2H, s), 2′/6′-6.90 ppm(2H, d), 3′/5′(2H, d)-7.85 ppm, 4′a-4.00 ppm(2H, s)

4-nitro-phenacyl thiazolium bromide (PTB-NO₂):

Thiazole (2.26 g) and 2-bromo-4′-nitroacetophenone (6.49 g) were combined in 30 mL anhydrous ethanol and refluxed for 2 hours during which a yellow, crystalline precipitate formed and recovered via filtration. The primary precipitate had a mass of 7.23 g (71% yield) and a melting point of 183° C.

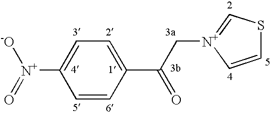

C-13 NMR peak/shift assignments:

C2-161.3, C4-138.4, C5-126.5, C1′-110.6, C2′-125.9, C3′-130.0, C4′-156.0, C5′-130.7, C6′-125.9, C3a-59.8, C3b-187.6

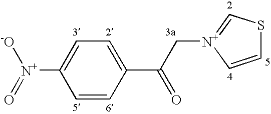

H NMR peak/shift assignments:

2-10.20 ppm, 4/5-8.40/8.50 ppm, 3a-6.30 ppm, 2′/6′-6.90 ppm, 3′/5′-7.85 ppm

Acetothiazole (AT):

Thiazole (1.0 g) and chloroacetone (1.1 g), 0.78M each, were combined in 15 mL of anhydrous ethanol and refluxed for 4 hours with stirring. A yellow/white precipitate was recovered via filtration and recrystallized in 95% ethanol generating 1.1 g purified AT (53% overall yield) with a melting point of 165° C.

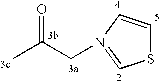

C-13 NMR peak/shift assignments:

C2-161.3, C4-138.0, C5-126.0, C3a-62.5, C3b-199.8, C3c-26.9

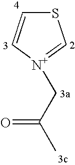

H NMR peak/shift assignments

2-10.25 ppm, 3/4 -8.35/8.50 ppm, 3a-5.85 ppm, 3c-2.25 ppm

1,3-dithiazole-2-propanone dibromide (DTPB):

Thiazole (1.58 g, 0.93M) and 1,3-dibromoacetone (2.0 g, 0.46M) were combined in 20 mL of anhydrous ethanol and refluxed for 4 hours. A brown precipitate was recovered from the reaction mixture by rotoevaporation of the ethanol. Recrystallization in 95% ethanol, and filtration yielded a tan/brown precipitate of mass 1.5 g (42% overall yield) with a melting point of >260° C.

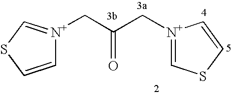

C-13 NMR peak/shift assignments

C2-157.6, C4-132.4, C5-109.9, C3a-61.8, C3b-206.0

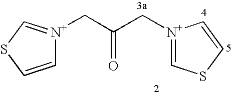

H NMR peak/shift assignments

3a(2H) 6.10 ppm, 2(1H) 10.25 ppm, 4/5 8.35/8.50 ppm

Thiamine and Thiamine Pyrophosphate:

Thiamine hydrochloride was purchased from Fisher Chemicals. Thiamine pyrophosphate chloride was purchased from ICN Biomedicals.

Results:

Referencing of Sigma and Alfa-Aesar Humic Acids (HA) to IHSS Humic Standards:

The complexity of naturally occurring materials produced in the environment results in significant variability in humic material characteristics between vendors and between lots from a single vendor that necessitates the comparison of commercially available humic acids to accepted humic acid standards. In this study, when samples were interrogated for humic acid concentrations via fluorimetry, known concentrations of commercial humic acids were compared to known concentrations of humic standards. Sigma-Aldrich humic acid (Sigma HA) and Alfa-Aesar humic acid (AA HA) solutions produce more intense fluorescence than the IHSS solutions under the same conditions of concentration by mass/volume, buffer composition and incident wavelength. All data presented in the accompanying figures have been referenced to IHSS standards via correction factors adjusting for differences in fluorescence intensities for these materials. Sigma HA and AA HA have factors of 2.2 and 7.5, respectively, greater fluorescence intensity than IHSS humic standard in concentrations generating linear fluorescence response. For the Sigma HA the linear fluorimetric response concentrations generally ranged 1-8 mg/L, for AA HA response is generally 1-4 mg/L and for IHSS HA it is typically 1-10 mg/L. All data presented in the accompanying figures were acquired in the linear response region appropriate for that material and then adjusted to IHSS values using the fluorescence correction factors.

Overview: Commercially Available Humic Acid Removal from Samples Via PIR Compounds

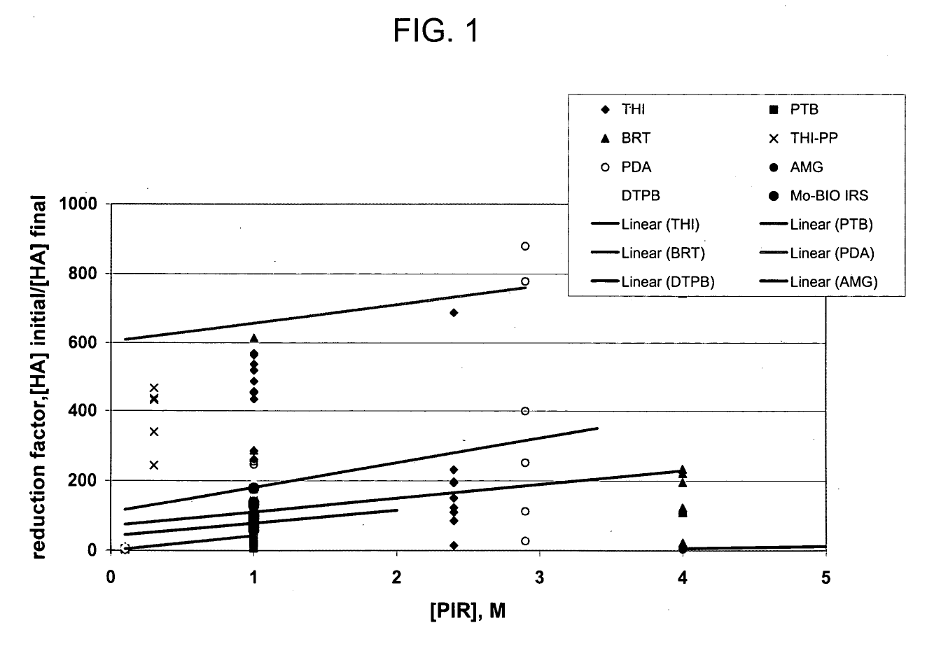

FIG. 1 compares the HA removal from samples containing DNA of interest to the molar concentration of varied PIR compounds by plotting an HA reduction factor as the initial [HA] divided by [HA] following treatment with PIR compounds. The commercially available PIR kit, from Mo-BIO, is placed on FIG. 1 for comparison and was arbitrarily assigned a molar concentration of 1 for convenience. The Mo-Bio IRS solution has been determined, via qualitative ICP-OES and semi-quantitative LC-MS, to principally contain aluminum sulfate. The data shown in FIG. 1 were generated from varied initial [HA], ranging from ˜5,000 to 300,000 mg/L HA. These varied initial [HA] values represent a “worst case” scenario and these are in comparison to initial [HA] in extractions from problematic soils generating humic acid concentrations typically less than 5,000 mg/L.

During the course of this study, many variables were incorporated in the PIR/PCR experiments and included: a) PIR compounds storage conditions (lighting, temperature and PIR compound concentration, b) humic/fulvic acid source, c) parent compound and parent compound analogues, and d) dilution factor or decade of dilution necessary for successful PCR. FIG. 1 is an overview of the humic acid data. No soil DNA/HA extraction data are presented in FIG. 1, only solution phase HA from commercial sources. HA data from soil extractions will be discussed separately.

Referring to FIG. 1, data for PIR compounds were plotted as points. Linear fits to these data points are also represented on the plot. Data corresponding to high reduction factors and low PIR compound concentration indicate better performing compounds. PIR compounds in decreasing order of HA removal efficacy based on HA reduction factors versus PIR compound molarity are: pyridoxamine (PDA)>thiamine (THI)>bromothiazole (BRT)>>1,3-dithiazole-2-propanone (DTPB)>the parent compound phenacyl thiazolium bromide (PTB); and finally, aminoguanidine (AMG). The Mo-BIO IRS solution's PIR efficacy falls between that of the parent compound PTB and THI.

Other PIR compounds were synthesized and characterized, or acquired, but are not included on this plot. These PIR compounds aminated phenacylthiazolium bromide(PTB-A), nitrophenacylthiazolium bromide (PTB-NO2), acetothiazole (AT) and thiamine pyrophosphate (THI-PP) did not perform well as PIR compounds, were not examined in time-storage studies, and will be discussed separately. Positive (CNT) and negative (NON) controls were also performed, relative to HA removal efficacy, and will be discussed separately; however, a broad perspective reveals that HA samples not treated with PIR compounds (NON) do not show any appreciable decrease in HA concentrations after being processed for PCR and, similarly, samples containing no added HA (CNT) do not show any appreciable measurement for HA or negative effects on PCR success.

A plot of HA reduction versus PIR compound concentration (FIG. 1) does not, of itself, indicate a compound's true value as a PIR compound. Consideration of dilution factors for successful PCR must also be reviewed. In untreated soil extracts containing DNA of interest, successful PCR of the extracted DNA may occur when the polymerase inhibitor is diluted sufficiently such that inhibition is suppressed and PCR may occur. Typical values for the dilution decade at which PCR is successful in samples containing humic materials and DNA of interest are often around decade four (1×10⁴). Accordingly, a dilution factor of 10,000 would be necessary for successful PCR in a typical soil extract/sample. This magnitude of dilution may also impact PCR sensitivity for DNA/species already present at low concentrations and result in not identifying an organism that was present in the original sample extract.

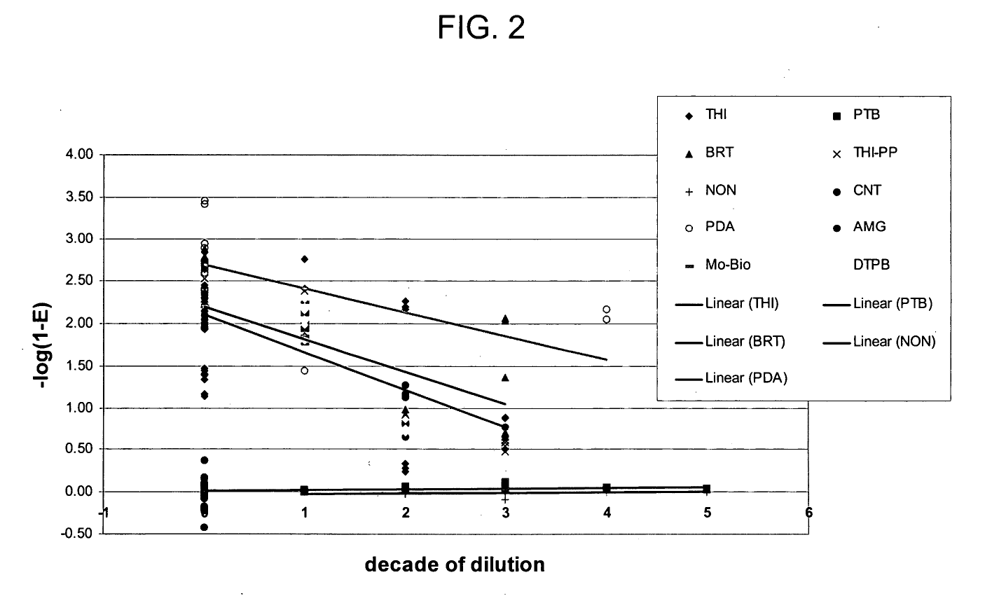

FIG. 2 plots the data from the same experiments represented in FIG. 1, but applying a different approach for data metric. FIG. 2 plots a dimensionless HA removal efficiency (or efficacy) value versus the decade of dilution necessary for successful PCR. The efficiency of HA removal is defined in equation 1 as the initial [HA] minus the final [HA] divided by the initial [HA]. This is essentially a percent removal approach.
Efficiency=[HA]ᵢ−[HA]_{f}/[HA]; (1)

To further discriminate efficiency values greater than 0.99, the efficiency was subtracted from the integer value of 1, and the −log was taken of 1-efficiency to generate a non-negative value indicative of HA removal efficiency with greater level of discrimination and resolution of values with high degrees of HA removal. This is described by equation 2.
0=−log (1-Efficiency) (2)

Using this approach for presenting the HA removal data allows for the determination of a PIR compound's effectiveness for HA removal (0) and compatibility for application in PCR systems via dilution data for PCR. Review of this approach reveals that data with high 0 values and low dilution decade values would come from a desirable PIR compound.

Similar to the results presented in FIG. 1, the data shown in FIG. 2 reveals that, in decreasing order of removal efficacy based on 0 and decade of dilution considerations, the best performing PIR candidates are: PDA>THI>BRT>PTB>DTPB>AMG. The Mo-BIO IRS solution PIR efficiency lies between THI and PTB. Considering the results shown in FIGS. 1 and 2, based on HA removal characteristics and decade of dilution for successful PCR, the best PIR compound candidates are pyridoxamine, thiamine and bromothiazole, in order of decreasing PIR efficacy. However, these two case considerations (reduction factors/PIR molarity and 0/decade of dilution) do not fully inform the PCR practitioner of the applicability of the PIR compound for HA removal in problematic samples. The quality and quantity of nucleic acid available for amplification by PCR following PIR treatment must also receive consideration. Quantitation of nucleic acid present in samples entering the PCR protocol is difficult at best. Determining the quality of nucleic acid is also difficult.

In general terms, the results of PCR amplification of the E. coli DNA used in the studies for HA removal reveal that pyridoxamine (PDA) and bromothiazole (BRT) were each problematic for DNA quantity and/or quality going into the PCR. Typically, PCR products were imaged after agarose gel electrophoresis in lanes of PCR product at dilution factors of 1 (no dilution) when using PDA; however, the intensity of the PDA treatment “band” was often low. Bromothiazole (BRT) had a similar effect on DNA. This can be interpreted as the PDA and/or BRT either: a) reduced the DNA concentrations/quantities present in samples via some chemical interaction or, b) reduced the DNA quality via some chemical interaction or, c) interacted with the DNA such that PCR amplification was unduly affected. Regardless of the mechanism for reduced DNA quality or quantity, in our samples tested with PIR compounds PDA and BRT are detrimental to the overall goal of the study. This result leaves thiamine (THI) as the best performing PIR compound tested when considering HA removal efficiency and dilution factors necessary for successful PCR.

Effects of Storage Conditions on PIR Compound Effectiveness:

The impact of storage conditions on PIR compound effectiveness were investigated, and included variations of temperature and lighting relative to days of storage for differing molarities of PIR solutions.

Phenacyl Thiazolium Bromide (PTB)

In earlier studies, the effectiveness of a PTB solution in improving PCR results from HA containing samples was observed to change with time. It was determined that investigating the lighting and temperature storage conditions might generate information regarding PTB's potential for photo or thermal degradation when stored in solution. Storage conditions for 0.1 and 1M PTB solutions were investigated.

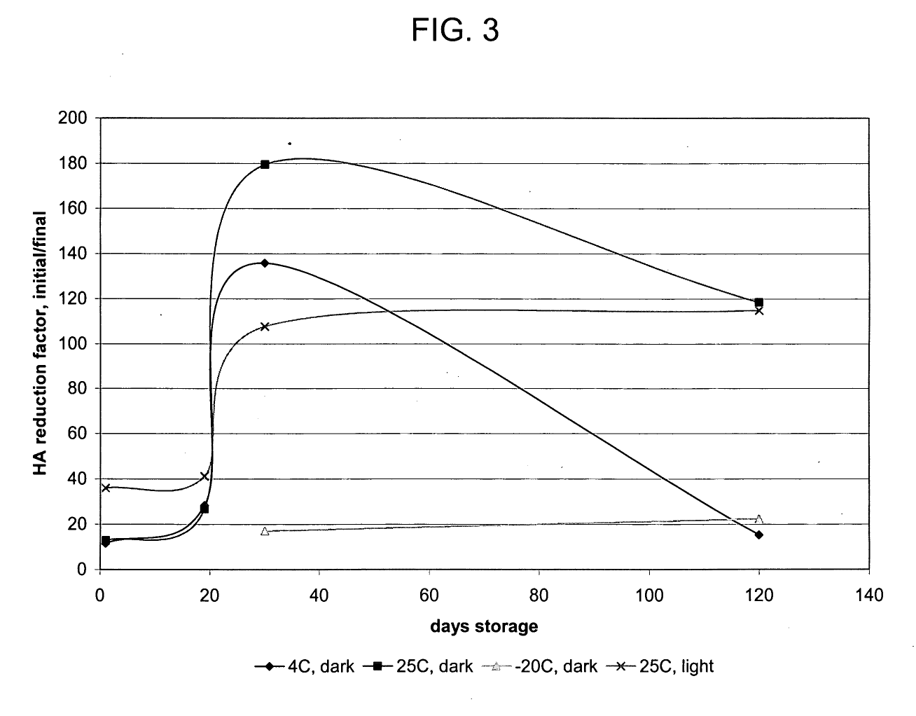

FIG. 3 plots the HA reduction factor of 1M PTB stored in phosphate buffer at −20° C. temperature and in darkness, 4° C. in darkness, 25° C. in darkness, and 25° C. under ambient laboratory lighting conditions (fluorescent lights). Initial HA concentrations averaged 10,200 mg/L ±1400. Unexpectedly, the 1M PTB stored below freezing and dark conditions (−20, dark) appears to have significantly decreased PIR ability. Following 120 days storage at low temperature both the 4° C. and −20° C. PIR solutions return to approximately the same PIR capability. Similarly, the samples stored at room temperature, regardless of lighting conditions, return to approximately the same PIR capability. Overall, the trend of PIR ability for PTB increases dramatically between solution preparation (day 1) and one-month storage, regardless of storage conditions, followed by slow decrease in PIR ability between 1-4 months. The storage conditions of stock solutions of phenacyl thiazolium bromide should be considered when applying PTB as a PIR agent and the mechanism for decreasing and increasing PIR ability, relative to storage conditions, should be further investigated.

0 values and decade of dilution for the 30 days storage experiments shown in FIG. 3 relative to 1M PTB solutions are in Table 1. Similar to examining the PIR efficiency via reduction factors, review of this data for 0 (effectiveness) and dilution decade reveals that even though similar effectiveness is achieved for all solutions stored above freezing, the room temperature storage conditions favor successful PCR products from samples containing DNA of interest, polymerase inhibitors and treated via our PIR protocols. PTB solutions of 0.1M show similar response to storage conditions as for 1M PTB solutions.

Based on these results, PTB solutions should be prepared in advance and used ˜20-40 days after preparation and stored at RT under ambient lighting.

TABLE 1
Ambient temperature storage allows PCR without dilution using 1M PTB.
30 days storage of 1M PTB in phosphate buffer

| Storage conditions | 0 values | Decade of dilution |
|---|---|---|
| 25 C., light | 2.0 | 0 |
| 25 C., dark | 2.2 | 0 |
| 4 C., dark | 2.1 | 1 |
| −20 C., dark | 1.2 | 1 |

Thiamine hydrochloride (THI)

Due to changes in PTB's PIR efficacy relative to time and storage conditions, THI was also investigated under similar conditions. Thiamine solutions of 0.1, 1 and 2.4M were stored at different temperature and lighting conditions and tested for PIR efficacy at 1, 19, 30, 120 and 180 days of storage. Initial HA concentrations averaged 10,200 mg/L ±1400.

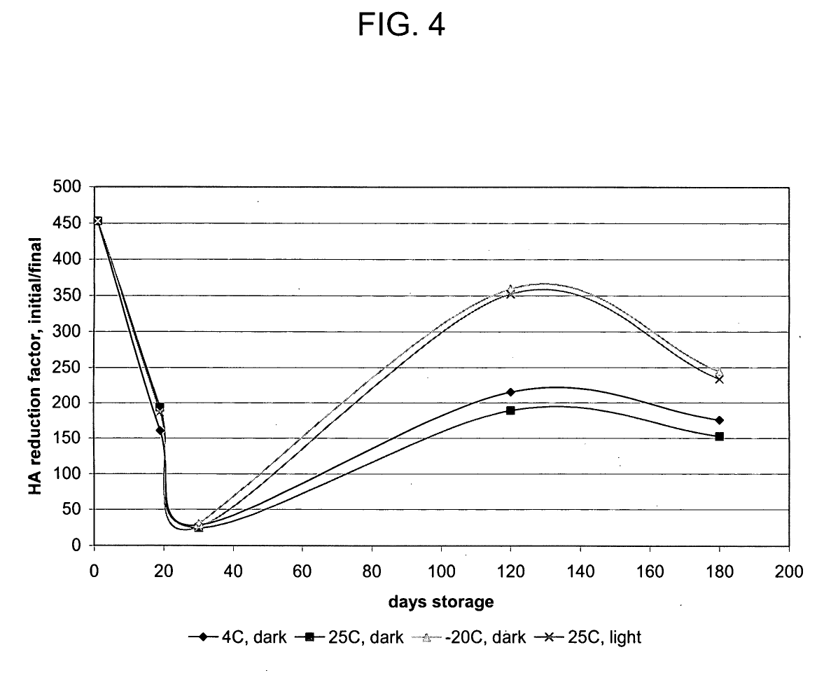

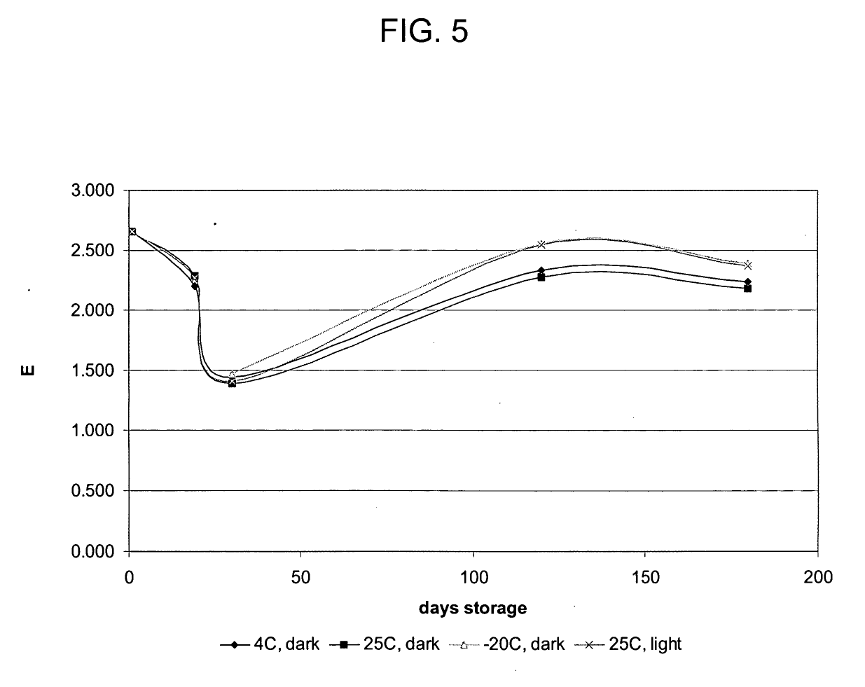

FIG. 4 illustrates the relationship of HA reduction factors and days of storage for 1M THI solutions stored under varying conditions of temperature and lighting. Similar to effects observed for PTB, thiamine's PIR efficacy is impacted by length of time in storage; however, there is no lighting condition dependency or relationship revealed. Thiamine's PIR reduction factors, for 1M THI, decrease between 1 and 30 days of storage, rebound and improve between days 30 and 120, and slightly decrease between 120 and 180 days. These fluctuations in PIR reduction factors do not appear to be influenced by the temperature or lighting conditions. Trends for PIR reduction factors for storage at −20° C./dark and 25° C./light are essentially the same. Similar trends are observed for storage at 4° C./dark and 25° C./dark, with the latter appearing to slightly under-perform relative to the former. FIG. 5 plots the data from the same experiments presented in FIG. 4; however, using 0 values and days of storage for comparison. The same trends are observed (decrease and rebound in PIR) with slightly smaller differences in the magnitude of PIR efficiency values (0 versus reduction factor). Solutions of THI at 0.1 and 2.4 M were also examined and followed the same trend patterns.

Based on these results, THI is best used as a freshly prepared solution instead of making up bulk stock solutions for long term storage and subsequent application as a PIR compound.

Bromothiazole (BRT)

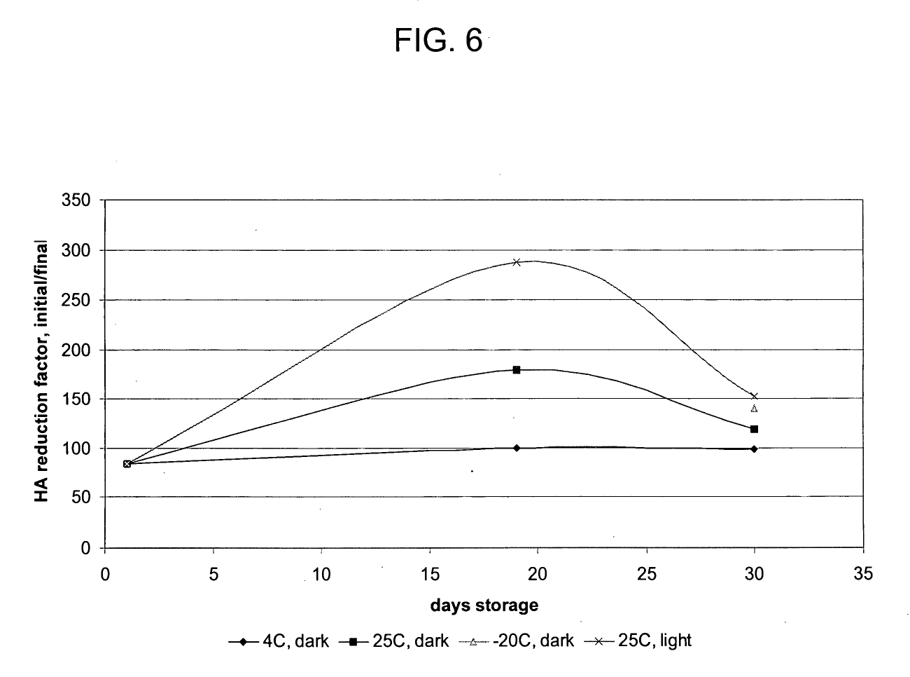

Solutions of 0.1, 1 and 4M bromothiazole were stored under the same conditions as PTB and THI described previously and tested for PIR efficacy. FIG. 6 plots the HA reduction factor versus days of storage (1, 19 and 30 days) under varied storage conditions of a 1M BRT phosphate buffered solution. HA concentrations averaged 10,200 mg/L ±100. As observed with PTB, there is an indication that storage at room temperature is favorable over colder storage, regardless of lighting conditions. The BRT solutions stored at room temperature have increasing PIR ability after 19 days of storage; however, when tested at day 30, the reduction factors do not vary widely between the solutions stored at RT or colder, regardless of lighting conditions. Solutions of 1M BRT stored at room temperature and exposed to ambient lighting appear to have increased PIR ability by a factor of ˜3 on day 19, versus day zero. Similarly, solutions of 1M BRT stored at room temperature, but kept in the dark, appear to have increased PIR ability by a factor slightly greater than 2 on day 19. All solutions rebound to approximately the same level of PIR ability by storage day 30.

Table 2 lists efficiency values for the experimental data in FIG. 6 on day 19 of storage. The higher 0 values for 1M BRT stored at room temperature, as well as greater HA reduction factors revealed in FIG. 6, show that solutions of 1M BRT may be stored at 25° C., without reducing the ability to perform as a PIR.

TABLE 2
1M BRT in phosphate buffer performs better when stored at RT
19 days storage of 1M BRT in phosphate buffer

| Storage conditions | 0 values | Decade of dilution |
|---|---|---|
| 25 C., light | 2.18 | 0 |
| 25 C., dark | 2.45 | 0 |
| 4 C., dark | 1.99 | 0 |
| −20 C., dark | 1.36 | 0 |

Pyridoxamine (PDA)

The effect of temperature and lighting storage conditions for pyridoxamine (PDA) solutions of 2.9 and 1.0M were examined after 30 days storage in −20° C./dark, 4° C./dark, 25° C./dark and 25° C./light. Table 3 lists HA reduction factors, HA 0 (effectiveness) and decade of dilution data for 2.9 and 1.0 M PDA solutions after 30 days storage under varied temperature and lighting conditions. HA concentrations averaged ˜10,000±100 mg/L for these experiments. The concentrated PDA solutions (2.9 M) have decreased PIR effectiveness when stored at RT. In contrast, the 1M PDA solutions do not display the same temperature dependency for any of the metrics (0 values, decade of dilution or HA reduction factors). Corresponding metrics for the 2.9M PDA solutions, that were freshly prepared (Day 0) are 2.6, 0 and 400 for 0 values, decade of dilution and HA reduction factors respectively. Without further investigation or verification of temperature effects on PIR ability of PDA, concentrated solutions of PDA preferably should not be stored at RT.

TABLE 3
Concentrated PDA solutions (2.9M) should be stored under low
temperatures

| | | | |
|---|---|---|---|
| Storage | | | HA reduction |
| conditions | 0 values | Decade of dilution | factor |

30 days storage of 1M PDA in phosphate buffer

| | | | |
|---|---|---|---|
| 25 C., light | 2.74 | 0 | 553 |
| 25 C., dark | 2.83 | 0 | 675 |
| 4 C., dark | 2.86 | 0 | 731 |
| −20 C., dark | 2.34 | 0 | 221 |

30 days storage of 2.9M PDA in phosphate buffer

| | | | |
|---|---|---|---|
| 25 C., light | 1.90 | 1 | 79 |
| 25 C., dark | 1.44 | 1 | 28 |
| 4 C., dark | 2.94 | 0 | 879 |
| −20 C., dark | 2.89 | 0 | 778 |

Aminoguanidine (AMG)

The effect of temperature and lighting storage conditions for aminoguanidine (AMG) solutions of 5.5 and 4.0M were examined after 30 days storage in −20° C./dark, 4° C./dark, 25° C./dark and 25° C./light. HA reduction factors, 0 values and decade of dilution values are listed in Table 4 and average HA concentrations for AMG-PIR experiments were ˜10,000±100 mg/L. As a PIR compound, AMG does not have as great HA removal ability as observed for THI, PDA, BRT or the parent compound PTB, regardless of the concentration of AMG. At both 4.0 and 5.5 M AMG concentrations all PIR-AMG experiments produced samples allowing for successful PCR products, but required dilution by 2 orders of magnitude (e.g. a dilution value of 2). Lighting or temperature appears to have no effect on the PIR ability of AMG.

Typical HA reduction factors for AMG are less than 20; this is in contrast with THI where reduction factors are 1-2 orders of magnitude greater. Similarly, 0 values for AMG are less than 1 for 4.0M AMG and less than 1.3 for 5.5 M AMG. These 0 values for AMG are contrasted by substantially greater 0 values shown for THI, BRT or PDA; however, AMG does appear to slightly outperform the parent compound PTB when comparing the three metrics shown in Table 4. Nonetheless, AMG underperforms as a PIR relative to other compounds studied.

TABLE 4
AMG has low HA reduction factors, 0 values, and typically requires
two orders of magnitude dilution for successful PCR.

| | | | |
|---|---|---|---|
| Storage | | | HA reduction |
| conditions | 0 values | Decade of dilution | factor |

30 days storage of 5.5 M AMG in phosphate buffer

| | | | |
|---|---|---|---|
| 25 C., light | 1.13 | 2 | 19 |
| 25 C., dark | 1.16 | 2 | 15 |
| 4 C., dark | 1.15 | 2 | 14 |
| −20 C., dark | 1.28 | 2 | 19 |

30 days storage of 4.0 M AMG in phosphate buffer

| | | | |
|---|---|---|---|
| 25 C., light | 0.80 | 2 | 6 |
| 25 C., dark | 0.81 | 2 | 6 |
| 4 C., dark | 0.83 | 2 | 7 |
| −20 C., dark | 0.65 | 2 | 4 |

31,3-dithiazolium-2-propanone (DTPB)

Solutions of 2, 1 and 0.1M DTPB were stored for 30 days with varied lighting and temperature conditions as described previously. Table 5 lists 0 values, HA reduction factor and decade of dilution for PCR products for these solutions. Regardless of DTPB concentrations, no PCR products were realized without dilution. Average HA concentrations were 50,300 mg/L±87. This initial HA concentration was approximately a factor of five greater than that for the other PIR storage condition experiments and the initial HA should be considered when comparing efficacy of PIR. An examination of the reduction factors and 0 values for DTPB listed in Table 5 reveals that 1.0M DTPB may be optimal compared to the higher concentration (2M) and lower concentration (0.1M) solutions. Further, there is an apparent slight advantage to storing DTPB solutions at RT versus colder temperatures, but this advantage is not manifested when considering decade of dilution necessary for PCR.

TABLE 5
0 values, HA reduction factors and decade of dilution data for 2.0, 1.0,
0.1 M DTPB solution in phosphate buffer stored for 30 days

| | | | |
|---|---|---|---|
| Storage | | | HA reduction |
| conditions | 0 values | Decade of dilution | factor |

30 days storage of 2M DTPB in phosphate buffer

| | | | |
|---|---|---|---|
| 25 C., light | 2.03 | 1 | 107 |
| 25 C., dark | 1.99 | 1 | 99 |
| 4 C., dark | 1.81 | 1 | 64 |
| −20 C., dark | 1.70 | 1 | 50 |

30 days storage of 1.0M DTPB in phosphate buffer

| | | | |
|---|---|---|---|
| 25 C., light | 2.29 | 1 | 194 |
| 25 C., dark | 2.20 | 1 | 160 |
| 4 C., dark | 2.16 | 1 | 143 |
| −20 C., dark | 2.06 | 1 | 114 |

30 days storage of 0.1M DTPB in phosphate buffer

| | | | |
|---|---|---|---|
| 25 C., light | 0.75 | 2 | 6 |
| 25 C., dark | 0.76 | 2 | 6 |
| 4 C., dark | 0.70 | 2 | 5 |
| −20 C., dark | 0.87 | 2 | 7 |

Summary of Storage Studies for PIR Compounds

The removal of commercial humic materials by PIR compounds was examined by comparisons of HA reduction factors ([HA]ᵢₙᵢₜᵢₐₗ/[HA]_{final}), efficiency of HA removal (0 values) and the magnitude of dilution necessary to achieve PCR following PIR treatment. Evaluation of PIR compounds by these metrics reveals that the commercially available compound, Thiamine hydrochloride (a Vitamin B), outperforms all other compounds tested and is considered the best PIR compound. In the practice of the methods of the invention, storage of thiamine solutions over time may be detrimental to overall efficacy as a PIR compound, and solutions of thiamine should therefore be prepared and consumed in as short a time frame as practical.

Pyridoxamine, a commercially available material, and bromothiazole (a synthetically available salt/PIR compound), each performed well in the removal of humic acids; however, unacceptable interactions with the DNA of interest may preclude the application of PDA or BRT as optimal PIR compounds. However, the greater degree of HA removal by PDA and BRT, relative to THI or the parent compound PTB, should be examined in further detail. If the mechanism of interaction between DNA and PDA/BRT can be determined, it may be possible to design analogues of PDA, or BRT, that have similar HA removal efficacy but reduced interactions with the DNA of interest.

Comparisons between the PIR compounds tested in these studies with a commercially available kit (MoBio) show that while the MoBio Soil DNA Kit protocols remove humic materials and allow for PCR products at dilution values similar to THI/PDA/BRT, the complexity of the MoBio protocol is greater than that for the PIR compounds tested in this study. Also, qualitative analysis of the MoBio IRS (Inhibitor Removal Solution), the active component of the MoBio Soil DNA kit, revealed that HA removal is likely due to chemical co-precipitation with aluminum as an aluminum oxyhydroxide floc. The co-precipitation of HA via aluminum, coupled with the need for spin filters, membrane binding and subsequent membrane washing, is a more laborious and technically demanding protocol than the one used in these studies.

The poor performance of aminoguanidine (AMG), 1-thiazole-2-propanone bromide (BAT, bromoacetothiazole), 4-amino-phenacylthiazolium bromide (PTB-A), 4-nitro-phenacylthiazolium bromide (PTB-N), thiamine pyrophosphate chloride (THI-PP), and 1,3-dithiazole-2-propanone (DTPB, dithiazole propanone bromide) relative to thiamine hydrochloride (THI) and pyridoxamine (PDA) precluded their incorporation into further studies of PIR ability on local soil samples which produce extracts problematic to PCR amplification of seeded E. coli DNA.

Solution Phase (Non-Soil Extracted) Humic Acid Removal via PIR Compounds:

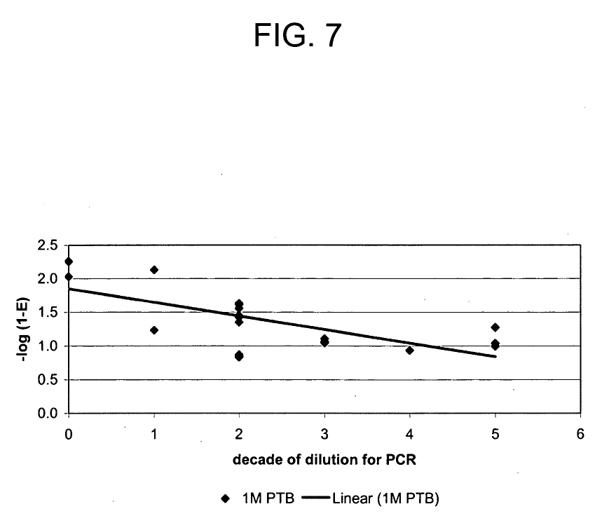

Phenacylthiazolium bromide (PTB) was investigated for its PIR efficacy in aqueous samples seeded with E. coli DNA and containing an average of 9100 mg/L humic acid (±600) initially. PTB, at 1M in phosphate buffer, had an average humic acid (HA) reduction factor of 43, at these initial HA concentrations, over 22 experiments. FIG. 7 plots efficiency values (0) versus the decade of dilution necessary to achieve PCR using 1M PTB for these 22 samples. In general, PTB treated samples required 1-2 orders of magnitude dilution to overcome any remaining PCR inhibition following treatment. Three samples allowed PCR without requiring any further dilution; however, these three samples had initial HA concentrations of 8200, 7700 and 8100 mg/L, which are lower than the average [HA]ᵢₙᵢₜᵢₐₗ of 9100 mg/L. Regarding these three experiments, no other PTB PIR experiments had lower initial humic acid concentrations and this result may indicate a threshold HA value for 1M PTB applied as a PIR compound. The reason for a large variation in decade of dilution to achieve PCR and efficiency values for 1M PTB is not certain. Possible factors include approaching, or exceeding, threshold HA values addressable via 1M PTB, differences in different humic materials tested (Sigma-Aldrich and Alfa Aesar) and/or Taq polymerase degradation. While decade of dilution for PCR varied over several orders of magnitude, HA reduction factors and 0 values did not have as significant of variation and this realization lends credence to reagent (polymerase) differences or experimental protocol uncertainty as the source of variability in decade of dilution results.

Table 6 lists PIR compounds investigated, PIR compound molarity, average initial humic acid concentrations for those reactions, average humic acid reduction factors, average efficiency values 0 (0=−log(1−E)) and typical dilution requirements to achieve PCR for aqueous samples of humic materials seeded with E. coli DNA. Regarding the decade of dilution necessary to achieve PCR; thiamine, bromothiazole and thiamine pyrophosphate were the PIR compounds that readily generated samples amenable to direct PCR without requiring further dilution to eliminate PCR inhibition. This is contrasted by the other PIR compounds which often required further dilution of the PIR treated sample in order to further remove PCR inhibition. The commercial product, MoBio's Soil DNA Kit, did perform well; however, this kit/protocol also required further dilutions. DTPB and BAT treated samples never achieved PCR products without requiring further dilution.

A review of average efficiency (0=−log (1−E)) values shows that PDA has significantly greater efficacy for HA removal and reasonable PCR dilution requirements; however, PCR products, as imaged after gel electrophoresis, from PDA treated samples were often “weak” as were products from BRT treatments.

Thiamine pyrophosphate was excellent at removing HA from aqueous systems, as revealed by examining reduction factors and efficiency values; however, its limited solubility required significant effort to achieve 0.3M solutions and the phosphate/pyrophosphate moieties may have interfered with the polymerase chain reaction.

TABLE 6
List of PIR compounds, concentrations, HA and HA
removal metrics and dilution data show that analogues
typically outperform parent compound PTB

| | | | | | Typical |
|---|---|---|---|---|---|
| | | | | | dilution |
| | | | | | decade |
| | | | Avg | | for PCR and |
| PIR | | HAᵢ, | Reduction | Average | (range of |
| compound | PIR, M | avg | Factor | Efficiency, 0 | dilutions) |
| PTB | 1* | 9,100 | 44 | 1.37 | 2 (0-5) |
| THI | 1 | 10,800 | 258 | 2.13 | 0 (0-3) |
| PDA | 1 | 12,300 | 251 | 4.80 | 1 (0-2) |
| BRT | 1 | 10,000 | 199 | 2.21 | 0 (0-3) |
| THI-PP | 0.3* | 14,000 | 384 | 2.57 | 0 (0-3) |
| MoBio IRS | n/a | 13,000 | 109 | 2.02 | 1 (0-1) |
| DTPB | 1 | 6,500 | 153 | 2.18 | 2 (1-2) |
| BAT | 1 | 9600 | 3 | 0.51 | 4 (3-4) |
| AMG | 4 | 35,000 | 6 | 0.77 | 4 (3-4) |

*maximum molar solubility of PTB and THI-PP.

Similar to the data generated in the storage condition studies, PIR efficacy based on efficiency values follows the general trend:

PDA>THI-PP>BRT>DTPB>THI>PTB>AMG>BAT.

Also similar, but based on reduction factors, PIR efficacy follows the trend:

THI-PP>THI>PDA>BRT>DTPB>PTB>AMG>BAT.

These trending schemes coupled with dilution decade for successful PCR show that the thiamine compounds THI/THI-PP and PDA are the primary PIR candidates for testing on soil extractions.

The disparity in initial HA concentrations for the AMG experiments (high HA), should have improved the efficiency and reduction factors for AMG while negatively impacting dilution decade at which PCR was successful. The low efficiency and reduction factor for AMG, regardless of initial HA concentration, eliminated this PIR from further testing in soil extracts. Similarly, BAT and DTPB were eliminated for further testing within soil extracts. The aminated parent compound (PTB-A) and nitrified parent compound (PTB-N) were also eliminated from further testing with soil extracts. The PTB-N was not stable in aqueous solutions and generated an orange/red precipitate approximately 1 hour following dissolution. Thiamine pyrophosphate (THI-PP) was not carried forward to testing on soil extracts due to its difficulty in dissolution and limited molar solubility of ˜0.3M; however, the pyrophosphate should be examined in further detail simply due to its considerable reduction factors and typical decade of; dilution. The aforementioned results generated a short list of PIR compounds (PDA, THI and PTB) for further testing with soil extracts.

Removal of Humic Acids from Soil Extracts via PIR Compounds:

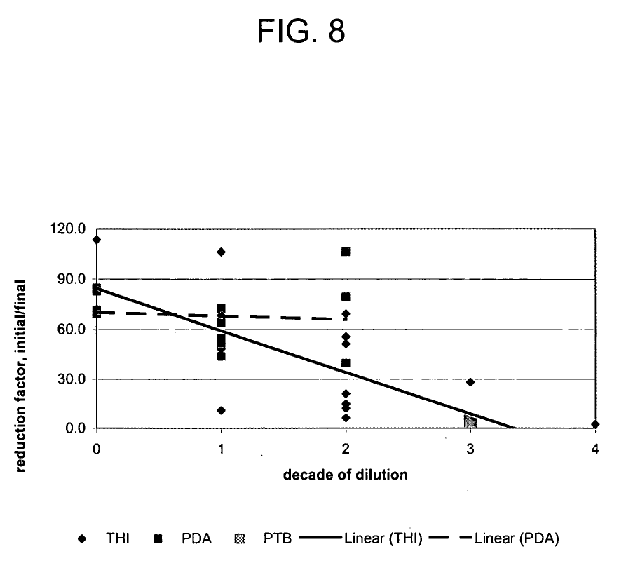

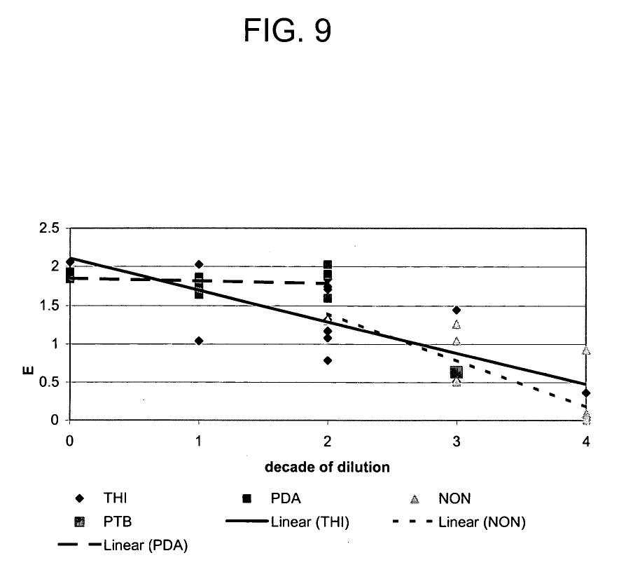

Thiamine and pyridoxamine were tested as PIR compounds in soil samples, and PTB was included for reference. Humic acid concentrations from soil extracts, referenced to IHSS humic acid, were 7,600 mg/L. FIG. 8 plots the HA reduction factor for THI, PDA, PTB and from a local, problematic soil (Los Alamos Airport Soil-LAAS). FIG. 9 plots the results from the same experiments but, using the efficiency metric instead of reduction factors. Both PDA and THI allowed for direct PCR, following PIR treatment, without requiring further dilution; however, this plot does not take into account varied PIR working concentrations. At 100 mM, the average reduction factors for THI and PDA were 45 and 68, respectively. At 100 mM, the average efficiency values for THI and PDA were 1.4 and 1.8 respectively. Examining the dilution decade at which PCR is achieved for the same conditions (100 mM PIR) shows that THI, on average, requires a dilution decade of 1.8 and PDA 1.0. While these performance criteria reveal that PDA is a better performing compound for humic acid removal, PCR products from PDA treated soil samples were typically weak when examined following gel electrophoresis and subsequent imaging for band intensity.

Compared to PTB, at 100 mM, reduction factor and efficiency values were 4 and 0.6. Further, PTB required further dilution of the treated sample by a factor of 1,000 in order to successfully achieve PCR.

### Example 2

### PIR Performance of Thiamine on Multiple Soil Types

This example provides an analysis of DNA extractions from multiple soil samples, using Thiamine as the PIR compound in the DNA extraction procedure, followed by nucleic acid amplification by PCR.

Materials and Methods:

A. DNA Extraction & Purification Procedure

Materials:

Sterile Prepared Bead Beater Tubes
10× PIR solution
1 ml syringes
0.2 μm pore size syringe filters, 13 mm diameter
1.5 ml eppendorf tubes
Microcon 100 filters, 500 μl volume
10 mM Tris pH 8.0
   Extraction:
1. Add ˜0.5 g soil to a prepared bead beater tube. (The soil and bead should consume ˜ half of the tube volume)
2. Homogenize 2 min in beadbeater (Biospec, Inc) at 3000 rpm.

Purification (Basic)
1. Add 100 μl of 10× PIR solution to homogenized soil.
2. Invert tube 4 times (minimum) to mix.
3. Centrifuge at 12,000 rpm (13,400×g) for 30 sec.
4. Draw supernatant into 1 ml syringe (avoid particulates).
5. Attach filter to syringe and filter supernatant into a sterile 1.5 ml Eppendorf.
6. Store at −20° C. or continue with extra purification.

Purification (Extra; Provides 10-Fold Increase Purity and Optional DNA Concentration)
1. Add 50-500 μl of DNA extract to Microcon 100 filter.
2. Place filter in collection tube and centrifuge at 7800 rpm (5600×g) for 3-8 minutes (until entire solution has passed through filter). Avoid excessive centrifugation.
3. (Optional wash step) Add 250 μl Tris solution (10 mM, pH8) to Microcon filter and centrifuge 4 minutes at 7800 rpm.
4. Add 50-500 μl Tris solution to Microcon filter. Invert filter in sterile Eppendorf tube and spin at maximum speed for 30 sec.
5. Store at −20° C.
   B. Beadbeater Tube Preparation:
   Materials:
b 2 ml screw cap tubes
0.1 mm beads (Biospec, Inc) and 0.5 mm beads (Biospec, Inc)
Sterile extraction buffer (TE pH8+0.1% SDS)
   Methods:
1. Add 0.22 g each of 0.1 and 0.5 mm beads to 2 ml tube
2. Loosely screw on cap (need to allow steam entry during autoclaving)
3. Sterilize by autoclaving 30 minutes
4. Add 900 μl sterile extraction buffer and store at room temperature.
   C. 10× PIR Solution:
100 mM NaPO₄ buffer pH7.4
PIR compound
1. dissolve PIR compound in NaPO₄ buffer to a final concentration of 1M
2. filter sterilize
3. use within 6 months
   D. Sodium Phosphate Buffer, 1M pH 7.4
For 1 liter:
43.46 g Na₂HPO₄
5.282 g NaH₂PO₄
Autoclave to sterilize
   Results:

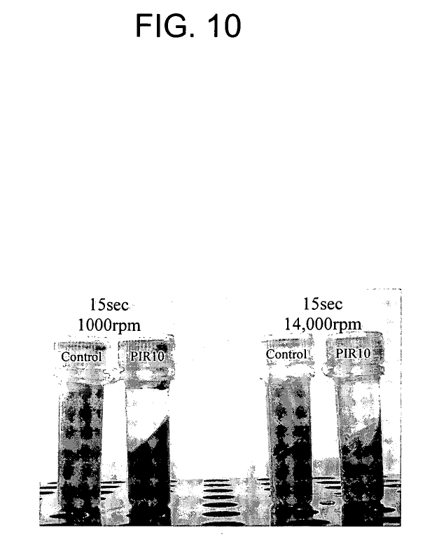

To test the ability of Thiamine to remove humic materials, an initial experiment designed to provide a visual demonstration of removal effectiveness was conducted as follows. A soil sample was homogenized (2 minutes), Thiamine was added to appropriate tubes, and all tubes were centrifuged at low speed (1000 rpm) and high speed (14,000 rpm). The results are shown in the images presented in FIG. 10. Even at low speed spins (1000 rpm), the PIR compound effectively removes particulates, indicating that this step can be replaced by coarse filtration.

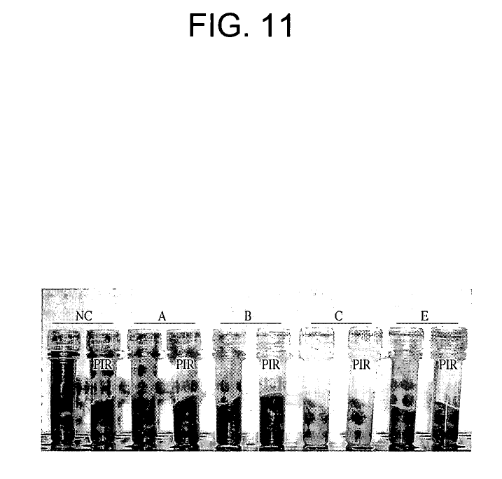

A further analysis of the PCR inhibitor removal effectiveness of Thiamine on a number of different soil types was then conducted. Crude extracts of the soil samples used in this experiment are shown in FIG. 11 to provide a visual illustration of the differences in humic acid content among the soil samples. All of these soil samplles were spiked with B. anthracis Sterne DNA such that the final concentration of Sterne DNA would be ≦100 pg/μl extract. Extractions were performed as described, supra.

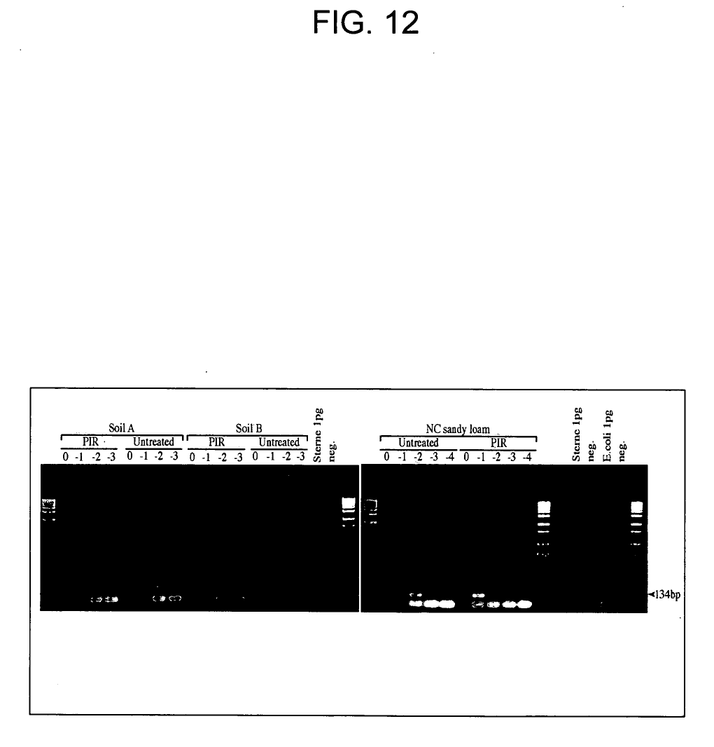

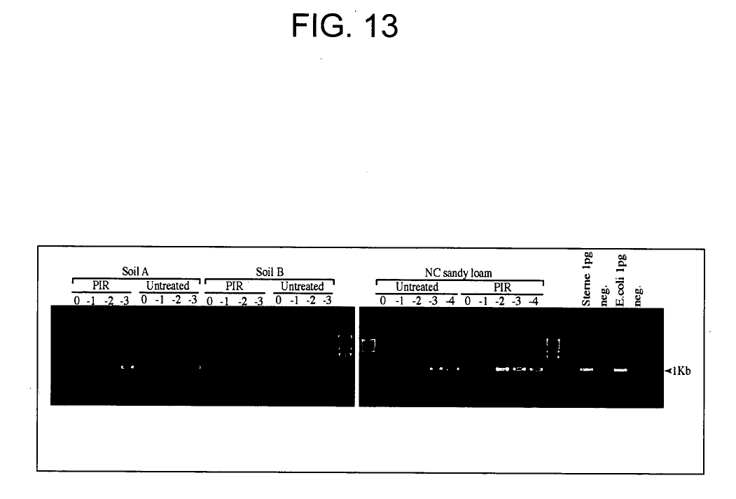

After purification, the soil extracts were serially diluted 10-fold in 10 mM Tris pH 8. To demonstrate DNA purity, a total of 60 PCR reactions were performed on all samples and dilutions for general amplication of 16S rDNA from bacterial DNA and for specific detection of B. anthracis Sterne in the samples. As shown in FIGS. 12 and 13, and and Tables 7 and 8, use of the PIR compound thiamine greatly improved detection of B. anthracis Sterne in the samples and also general amplification of 16S rDNA. The results with soils A and B were reconfirmed by repeating the extractions and PCR testing.

TABLE 7
Summary of B. anthracis PCR detection results with treated
& untreated DNA from spiked soils A, B, and the NC sandy loam.

| | | 1^{st} DNA dilution | |
|---|---|---|---|
| Soil | Treatment | with positive PCR | B. anthracis per rxn |
| soil “A” | Untreated | 10⁰, very faint | ˜10⁴ cell equivalents |
| | | product | |
| | PIR | 10⁰ | ˜10⁴ cell equivalents |
| soil “B” | Untreated | 10⁰, faint product | ˜10⁴ cell equivalents |
| | PIR | 10⁰ | ˜10⁴ cell equivalents |
| soil “C” | Untreated | 10⁻² | ˜10² cell equivalents |
| | PIR | 10⁰ | ˜10⁴ cell equivalents |

TABLE 8
Summary of general bacterial 16S rDNA PCR detection results
with treated & untreated DNA from soils A, B, and the NC sandy loam.

| | | 1^{st} DNA dilution |
|---|---|---|
| Soil | Treatment | with positive PCR |
| “A” | Untreated | 10⁻², fainter product |
| | PIR | 10⁻² |
| “B” | Untreated | 10⁻², fainter product |
| | PIR | 10⁻² |
| NC soil | Untreated | 10⁻³ |
| | PIR | 10⁻¹ |

Addition of Ultrafiltration to Basic Protocol:

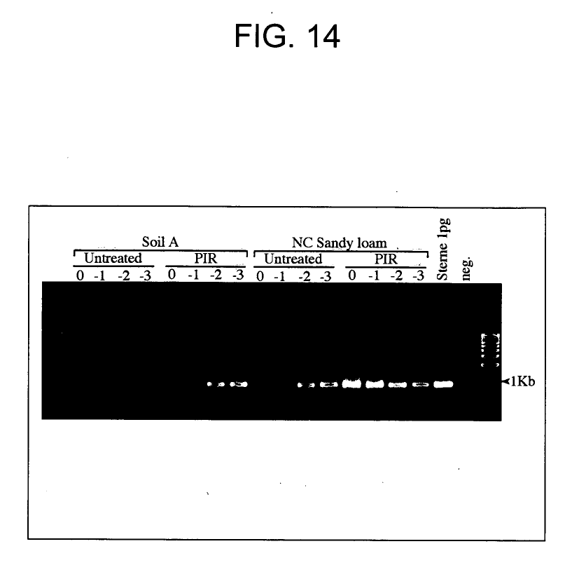

Soil A and the NC sandy loam soil extracts were subjected to ultrafiltration (i.e., filtration through a 100,000 kDa molecular weight cut-off filter). Ultrafiltration provides a means to remove inhibitory salts and metal ions from DNA extracts. As shown in FIG. 14 and Table 9, ultrafiltration increased the purity of the DNA samples about an order of magnitude.

TABLE 9
Summary of general bacterial 16S rDNA PCR detection results with
ultrafiltered DNA (treated and untreated) from soil A and the NC
sandy loam.

| | | DNA dilution with |
|---|---|---|
| Soil | Treatment | positive PCR |
| “A” | Untreated | 10⁻² |
| | PIR | 10⁻¹ |
| NC soil | Untreated | 10⁻² |
| | PIR | 10⁰ |

Note:
Comparison with results in Table 8 illustrates the extra 10-fold increase in DNA purity that generally occurs with ultrafiltration.

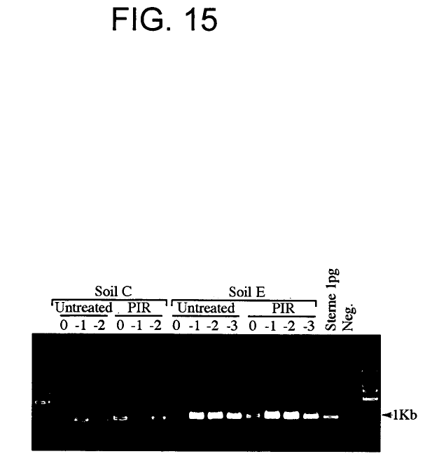

A further two additional soil samples were subjected to DNA extraction using Thiamine as the PIR compound, as follows. Soils C and E were processed using the basic purification protocol (not including ultrafiltration). General amplification of 16S rDNA from serial dilutions of the extracts are shown in FIG. 15. Similar results were obtained with both soils—treatment with the PIR compound produced at least a 10-fold increase in purity, enabling successful PCR without dilution of the soil extract (Table 7).

All publications, patents, and patent applications cited in this specification are herein incorporated by reference as if each individual publication or patent application were specifically and individually indicated to be incorporated by reference.

The present invention is not to be limited in scope by the embodiments disclosed herein, which are intended as single illustrations of individual aspects of the invention, and any which are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### LITERATURE CITED

1. Hilger, A.; Myrold, D.; “Method for Extraction of Frankia DNA from Soil”, Agriculture, Ecosystems and Environment, Vol. 34, 1991, p 107-113.
2. Ball, P.; Holben, W.; Woessner, W.; Gsell, T.; Deborde, D.; “Assessing Mass Balance of Transported Bacteriophage in a Septic Flowfield”, Abstracts of the General Meeting of the American Society for Microbiology, 99^{th} General Meeting, 1999, Chicago, Ill., USA.
3. Leff, L.; Dana, J.; McArthur, J.; Shimkets, L; “Comparison of Methods of DNA Extraction from Stream Sediments”, Applied and Environmental Microbiology, Vol. 61, No. 3, 1995, pp 141-1143.
4. Steffan, R.; Goksoyr, J.; Asim, B.; Atlas, R.; “Recovery of DNA from Soils and Sediments”, Applied and Environmental Microbiology, Vol. 54, 1988, p 2908-2915.
5. Tsai, Y.; Olson, B.; “Rapid Method for Separation of Bacterial DNA from Humic Substances in Sediments for Polymerase Chain Reaction”, Applied and Environmental Microbiology, Vol. 58, 1992, p 2292-2295.
6. Tsai, Y.; Palmer, C.; Sangermano, L.; “Detection of Escherichia coli in Sewage and Sludge by Polymerase Chain Reaction”, Applied and Environmental Microbiology, Vol. 59, 1993, p 353-357.
7. Alm, E; Zheng, D.; Raskin, L; “The Presence of Humic Substances and DNA in RNA Extracts Affects Hybridization Results”, Applied and Environmental Microbiology, Vol. 66, 2000, p 4547-4554.
8. Kuske, C.; Banton, K.; Adorada, D.; Stark, P.; Hill, K.; Jackson, P.; “Small-scale DNA Sample Preparation Method for Field PCR Detection of Microbial Cells and Spores in Soil”, Applied and Environmental Microbiology, Vol. 64., 1998, p 2463-2472.
9. Aiken, G. R.; In Humic Substances in Soil, Appendix A, 1985.
10. Chandler, D.; Schreckhise, R.; Smith, J.; Bolton, H.; “Electroelution for Removal of Humic Compounds from Soil DNA and RNA Extracts”, Journal of Microbiological Methods, Vol. 28, 1997, p 11-19.
11. Vescio, P.; Nierzwicki-Bauer, S.; “Extraction and Purification of PCR Amplifiable DNA from Lacustrine Subsurface Sediments”, Journal of Microbiological Methods, Vol. 21, 1995, p 225-233.
12. Berthelet, M.; Whyte, L.; Greer, C.; “Rapid, Direct Extraction of DNA from Soils for PCR Analysis using Polyvinylpolypyrrolidone Spin Columns”, FEMS Microbiology Letters, Vol. 138, 1996, p 17-22.
13. Khan, A.; Jones, R.; Cerniglia, C.; “Rapid. Method for the Detection of Genetically Engineered Microorganisms by Polymerase Chain Reaction from Soil and Sediments”, Journal of Industrial Microbiology and Biotechnology, Vol. 20, 1998, p 90-94.
14. Kreader, C.; “Relief of Amplification Inhibition in PCR with Bovine Serum Albumin or T4 Gene 32 Protein”, Applied and Environmental Microbiology, Vol. 62, 1996, p 1102-1106.
15. Watson, R.; Blackwell, B.; “Purification and Characterization of a Common Soil Component which Inhibits the Polymerase Chain Reaction”, Canadian Journal of Microbiology, Vol. 46, 2000, p 633-642.
16. Miller, D.; “Evaluation of Gel Filtration Resins for the Removal of PCR-Inhibitory Substances from Soils and Sediments”, Journal of Microbiological Methods, Vol., 44, 2001, p 49-58.
17. LaMontagne, M.; Michel, F.; Holden, P.; Reddy, C.; “Evaluation of Extraction and Purification Methods for Obtaining PCR-amplifiable DNA from Compost for Microbial Community Analysis”, Journal of Microbiological Methods, Vol. 49, 2002, p 255-264.
18. Braid, M.; Daniels, L.; Kifts, C.; “Removal of PCR Inhibitors from Soil DNA by Chemical Flocculation”, Journal of Microbiological Methods, Vol. 52, 2003, p 289-393.
19. Zhou, J.; Hurt, R.; Qiu, X.; Wu, L.; Roh, y.: Palumbo, A.; Tiedje, J.; “Simultaneous Recovery of RNA and DNA from Soils and Sediments”, Applied and Environmental Microbiology, Vol. 67, p 4495-4503.
20. Torsvik, V.; “Isolation of Bacterial DNA from Soil”, Soil Biology and Biochemistry, Vol. 12, 1980, p 15-21.
21. Meis, J.; Chen, F.; “The SoilMaster™ DNA Extraction Kti Provides PCR-Ready Soil DNA in Less Than an Hour”, Epicentre Forum, Vol. 9, 2002, p 1-3.
22. Meis, J.; Chen, F.; “Extract PCR-Ready Soil DNA in Less Than an Hour with the New SoilMaster™ DNA Extraction Kit”, Epicentre Forum, Vol. 9, 2003, p 1-3.
23. Kramer, F; Vollrath, T.; Schnieder, T.; Epe, C.; “Improved Detection of Endoparasite DNA in Soil Ssample PCR by the Use of Anti-Inhibitory Substances”, Veterinary Parasitology, Vol. 108, 2002, p 217-226.
24. Zoll, G.; Grote, G., Dierstein, R.; Kohne, S.; “Rapid Isolation of Anthrax DNA from Large-Volume Soil Samples Using QIAamp® Kits”, Qiagen News, Issue No. 1, 2002, p 22-23.
25. Holland, J.; Louie, L.; Simor, A.; Louie, M.; “PCR Detection of Escherichia coli O157:H7 Directly from Stools: Evaluation of Commercial Extraction Methods for Purifying Fecal DNA”, Journal of Clinical Microbiology, Vol. 38, 2000, p 4108-4113.
26. Vasan, S.; Zhang, X.; Zhang, X.; Kapurniotu, A.; Bernhagen, J.; Teichberg, S.; Basgen, J.; Wagle, D.; Shih, D.; Terlecky, H.; Bucala, R.; Cerami, A.; Egan, J.; Ulrich, P.; “An Agent Cleaving Glucose-derived Protein Crosslink in vitro and in vivo”, Nature, Vol. 382, 1996, p 275-278.
27. Ferguson, G.; VanPatten, S.; Bucala, R.; Al-Abed, Y.; “Detoxification of Methylglyoxal by the Nucleophilic Bidentate, Phenylacylthiazolium Bromide”, Chem. Res. Toxicol., Vol. 12, 1999, p 617-622.
28. Poinar, H.; Hofreiter, M.; Spaulding, W.; Martin, P.; Stankiewica, B.; Bland, H.; Evershed, R.; Possnert, G.; Paabo, S.; “Molecular Coproscopy: Dung and Diet of the Extinct Ground Sloth Nothrotheriops Shstensis”, Science, Vol. 281, 1998, p 402-405.
29. Kemp, B.; Smith, D.; “Tackling (some of) the vagaries of ancient DNA work”, News and Events, Trace Genetics LLC, 2003.
30. Vartzouma, C.; Louloudi, M.; Butler, I.; Hadjiliadis, N.; “Practical Tethering of Vitamin B1 on a Silica Surface via its Phosphate Group and Evaluation of its Activity”, ChemComm Communication, 2002, p 522-523.
31. Cras, J.; Taift-Rowe, C.; Nivens, D.; Ligler, F.; “Comparison of chemical cleaning methods of glass in preparation for silanization”, Biosensors & Bioelectronics, Vol. 14, 1999, p 683-688.
32. Vasan et al., 2003, “Therapeutic potential of breakers of advanced glycation end product-protein crosslinks”, Archives Biochem. Biophys. 419: 89-96.

What is claimed is:

1. A method for preparing a purified nucleic acid-containing extract from an environmental sample of soil, fluid, or organic particles, comprising:
(a) preparing an aqueous nucleic acid containing extract from the environmental sample;
(b) adding to the extract at least one PIR compound selected from the group consisting of thiamine hydrochloride, thiamine pyrophosphate, 1,3-dithiazole-2-propanone dibromide, and pyridoxamine;
(c) mixing the PIR compound(s) into the extract for a time sufficient to precipitate humic acids, fulvic acids and/or other insoluble contaminants contained in the extract, thereby generating insoluble precipitate and soluble fractions; and
(d) isolating the soluble fraction therefrom;

2. The method according to claim 1, further comprising subjecting the soluble fraction of step (d) to further nucleic acid purification.

3. The method according to claim 2, wherein further nucleic acid purification is achieved by precipitating nucleic acids within the soluble fraction, pelleting the precipitated nucleic acids, and resuspending the nucleic acids in a buffered solution.

4. The method according to claim 3, further comprising subjecting the resuspended nucleic acids to ultrafiltration.

5. The method according to claim 1, wherein the aqueous nucleic acid containing extract of step (a) is prepared by lysing cells or microbe particles contained in the sample by homogenization in the presence of a surfactant or detergent.

6. The method according to claim 5, wherein homogenization is achieved by vigorous mixing of the sample with glass, ceramic and/or metallic beads ranging from 0.1 to 0.5 mm in diameter, in the presence of SDS.

7. The method according to claim 6, wherein insoluble debris generated by homogenization is removed prior to the addition of the PIR compound in step (b).

8. A reagent kit for preparing a purified nucleic acid-containing extract from an environmental sample of soil, fluid, or organic particles, comprising: (a) at least one PIR compound slected from the group consisting of thamine hydrochloride, thiamine pyrophosphate, 1,3-dithiazole-2-propanone dibromide, and pyridoxamine; and, (b) instructions for using the PIR compound in accordance with the method of claim 1.
